(19) United States
(12) Patent Application Publication
(10) Pub. No.: US 2025/0387493 A1
(43) Pub. Date: Dec. 25, 2025

(54) METHOD OF ABROGATING ANTI-DRUG ANTIBODY FORMATION, AND DRUG CONJUGATES FOR USE THEREIN

(71) Applicant: OncoC4, Inc., Rockville, MD (US)

(72) Inventors: Yang Liu, Potomac, MD (US); Ido D. Weiss, Silver Spring, MD (US); Nabin Poudel, Gaithersburg, MD (US); Don Tong, Guangdong (CN)

(21) Appl. No.: 19/247,987

(22) Filed: Jun. 24, 2025

Related U.S. Application Data
(60) Provisional application No. 63/663,790, filed on Jun. 25, 2024.

(51) Int. Cl. A61K 47/55; A61K 49/00

(52) U.S. Cl. CPC A61K 47/552; A61K 49/0008

(57) ABSTRACT

The invention includes a method of treatment using a therapeutic agent for a condition which the therapeutic agent is effective. The method includes (a) at least one pre-treatment administration to a patient of a therapeutic drug conjugate, wherein the therapeutic drug conjugate is in the form of T-L-C, wherein T comprises the therapeutic agent, L comprises a linker and C comprises a cytotoxic agent; and (b) a first therapeutic administration to a patient of the therapeutic agent. The first therapeutic administration of the therapeutic agent is at a therapeutic level for treating the condition for which the therapeutic agent is effective and the at least one pre-treatment administration of the therapeutic drug conjugate is at a level that is below the therapeutic level for treating the condition with the therapeutic agent alone.

### CROSS REFERENCE TO RELATED APPLICATIONS

This application claims the benefit of priority of U.S. Provisional Patent Application No. 63/663,790, filed on Jun. 25, 2024, the entirety of which is incorporated herein by reference.

### REFERENCE TO A SEQUENCE LISTING

The application contains a Sequence Listing which has been submitted electronically in ST.26 Sequence listing XML format and is hereby incorporated by reference in its entirety. Said ST.26 Sequence listing XML, created on Jun. 19, 2025 is titled 8016_001_Sequence_Listing.xml and is 27,359 bytes in size.

### FIELD OF THE INVENTION

The disclosure relates to a method of abrogating the formation of anti-drug antibodies using therapeutic drug conjugates and compositions used therein.

### BACKGROUND OF THE INVENTION

Therapeutic proteins are an important tool for modern medicine; however, it is known in the art that repeated administration of these agents to patients can cause immunogenic responses. While recent advances in humanized and fully human monoclonal antibodies reduce the immunogenicity, it has not yet been eliminated. One manifestation of this immunogenicity is via the formation of anti-drug antibodies (“ADA”) by B-cells. These antibodies against therapeutic proteins, including monoclonal antibodies, enzymes, fusion proteins and protein replacement therapies, can develop in some patients receiving these treatments. The presence of such anti-drug antibodies can potentially impact the safety and efficacy of the therapy by altering its pharmacokinetic and pharmacodynamic properties, reducing its bioavailability, or causing serious adverse immunogenic reactions, such as anaphylaxis. In some cases, development of ADA in patients prevents further continuation of what would otherwise be an effective treatment regime.

The propensity for ADA development depends on various factors, such as the protein's structural characteristics, route of administration, treatment regimen, and patient-specific factors like genetic background and immunological status. There is currently no effective treatment against anti-drug antibodies once they are formed, so there is need for a method to prevent their formation in advance.

Many approved therapeutics are known in the art to generate ADA during the course of administration, including but not limited to Adalimumab, Alemtuzumab, Atezolizumab, Benralizumab, Cetuximab, Dinutuximab, Elotuzumab, Erenumab, Galcanezumab, Golimumab, Guselkumab, Infliximab, Ixekizumab, Ipilimumab, Lanadelumab, Natalizumab, Nivolumab, Obinutuzumab, Risankizumab, Rituximab, Romosozumab, Sarilumab, Tildrakizumab, Tocilizumab, Trastuzumab, Ustekinumab, Vedolizumab, myozyme, nexviazyme, Palynziq, krystexxa, and other recombinant proteins in replacement therapies. Antibodies against viral proteins are also barriers for gene therapy, while those against genetically modified cells may impede efficacy of cell therapy.

Anti-drug antibodies develop in patients whose B-cells express a B-cell receptor that recognizes the therapeutic. Upon such interaction of the B-cell receptor with the therapeutic, the B cells are activated, expand and differentiate into memory B cells and plasma cells, which produce high amounts of ADA. Elimination of therapeutic-specific B cells before the initial therapeutic exposure in a patient should lead to lasting immune tolerance which prevents the generation of ADA in the first place, therefore improving the exposure to the therapeutic and prolonging the time the therapeutic is effective. ADA not only reduce therapeutic activity of the drug, but also cause infusion reactions and possibly severe anaphylaxis.

Current methods for prevention of ADA production include depletion of B cells in an antigen non-specific way. Alternatively, therapeutic proteins were combined with general immune suppressant. Neither approach is effective and both increased risk of infection and cancer. To avoid broad immune suppression, repeated dosing with high dose therapeutic proteins has been shown to reduce ADA in some patients. However, such an approach requires high doses of therapeutic drug, extended dosing over several months, and is often not effective. The invention disclosed herein takes advantage of the fact that B cells responsible for the production of ADA-expressing clonal antigen-specific receptor can be targeted by the therapeutic proteins. Protein therapeutic-drug conjugates (TDC) are particularly suitable for this purpose as they will be endocytosed following binding to B cell antigen-receptor, enter into lysosomes for degradation and release the payload for the destruction of the antigen-specific B cells. Thus, coupling cytotoxic agents to the therapeutic proteins results in specific removal of ADA producing cells, leading to rapid induction of long-lasting immune tolerance to the therapeutic proteins.

Therapeutic-drug conjugates are based on the therapeutics that are used for treatment but are conjugated with the high-efficiency cytotoxicity of cytotoxic drugs. An example of such therapeutic-drug conjugates are antibody-drug conjugates, which consist of a monoclonal antibody conjugated via a suitable linker to an appropriate cytotoxic agent. Other therapeutic proteins can also be conjugated to appropriate cytotoxic agents. Furthermore, the antibody to drug moiety conjugation ratio (“DAR”) can also be controlled to have the desired effect.

Therefore, there is a need in the art for a method to prevent the generation of ADA in patients, thereby prolonging the use of therapeutics and improving their safety.

### DESCRIPTION OF THE DRAWINGS

FIGS. 1A-1C. ONC784-B-5 prevents ADA generation and increases ONC781 accumulation. FIG. 1A: Experimental design and timeline. FIG. 1B: Anti-ONC-781 ADA titers in the serum. FIG. 1C: Levels of ONC781 in the circulation at Day 18. CD24h/h mice, females, 6-12 weeks old, were injected either with ONC781, ONC784-B-5, or ONC784-B-7 at 15 mg/Kg dose intravenously twice weekly for a total of four injections. One week after the final injections, blood was collected and surveyed for anti-ONC781 ADA and circulating levels of ONC781.

FIGS. 2A-2D. ADA reduced drug accumulation and cancer therapeutic efficacy. FIG. 2A: Experimental design and timeline. FIG. 2B: Tumor growth kinetics of tumors in different groups. FIG. 2C: Anti-ONC781 ADA titers in the serum. FIG. 2D: Levels of ONC781 in the circulation (serum). CD24^{h/h} mice, females, 12-31 weeks old, were transplanted with MC38 tumor cells expressing human CD24 (MC38hCD24). Starting at day 12 after tumor inoculation, mice received either IgG, ONC784-B-5, or ONC784-B-7 at 5 mg/Kg dose intravenously twice weekly for a total of four injections. Blood samples were collected two weeks after the final injection and surveyed for anti-ONC781 ADA and circulating levels of ONC781. Tumor volumes were measured twice a week.

FIGS. 3A-3I. A short course of pretreatment with low dose of ONC784-B5 induced long-lasting immune tolerance to ONC784. FIG. 3A: Experimental model and timeline. Bold arrows indicate injections of either the candidate ADCs or ONC781. Narrow arrows indicate timepoint when blood samples were analyzed. FIGS. 3B-3E: Serum ADA titers on indicated days show that all tested dosage of ONC784-B-5 prevented ADA generation. The effect persisted for more than 74 days after initial intervention. ADA Titer 0 means not detected. FIGS. 3GF-3I: Levels of ONC781 in circulation on each day shows that the circulating ONC781 levels tended to be higher for ONC784-B-5 group compared to ONC781 group on D10. However, on D28, ONC784-B-5 injected group had a significantly higher levels of circulation ONC781 compared to ONC781 injected group indicating neutralization of injected mAbs by circulating ADA in the ONC781 treated group, and longer preservation of ONC781 in mice that lack ADA. CD24^{h/h} mice received either 250 μg of ONC-784 antibody or a range of titration of ONC784-B-5 (250 μg, 25 μg, and 5 μg) per mouse three days apart. Blood samples were analyzed for ADA titers and circulating levels of naked antibody one week after the second injection on day 10. Mice were then given intravenous injection of ONC781 at a dosage of 5 mg/Kg for four injections twice weekly starting day 11. Blood samples were surveyed for ADA, circulating B cells and circulating ONC781 after one week on day 28. Mice were then rested and given an additional injection of ONC781 at 5 mg/kg on day 52 (one month after the last injection on day 21). Blood samples were again surveyed for ADA and ONC781 in circulation after one week on day 58. Mice were given an additional injection of ONC781 at 15 mg/Kg on day 67 and serum was surveyed for ADA on D74.

FIGS. 4A-4C. A generally applicable method for identification and use of ADCs for preventing ADA generation and anaphylaxis. FIG. 4A: Experimental model and timeline. Bold arrows indicate injections of either the candidate ADCs or ONC781. Narrow arrows indicate timepoints when blood samples were analyzed (Day 19) or mice were evaluated for hypersensitivity (Day 20). FIG. 4B: Serum ADA titers on day 19. ADA Titer 0 means not detected or similar levels as Naïve serum. FIG. 4C: Clinical score for mice evaluated at day 20. Female C57BL/6 mice were pretreated with 5 μg of naked antibody or candidate ADCs three days apart followed by an intravenous injection of ONC-781 at a dosage of 5 mg/Kg on day 12. Blood samples were surveyed for ADA after one week.

FIG. 5. Binding of Adalimumab and its variant ADM-A-800 to TNFα. The data shows that ADM-A-800 has no detectable binding to human TNF-alpha in an ELISA assay.

FIGS. 6A-D. Adalimumab and adalimumab ADCs bind similarly to TNF-alpha while adalimumab variant ADCs do not. The data shows the ELISA binding comparison for adalimumab, adalimumab ADC (ADM-A-003-B5), and adalimumab variant ADCs (ADM-A-800-B5 and ADM-A-800-B8) to coated (FIG. 6A, FIG. 6B) Human TNF-alpha and (FIG. 6C, FIG. 6D) Mouse TNF-alpha.

### DETAILED DESCRIPTION OF THE INVENTION

The inventors have determined that it is possible to abrogate the level of ADA to a particular known therapeutic agent in patients by administering a therapeutic drug conjugate (“TDC”), such as an anti-body drug conjugate (“ADC”), to a patient. The term, therapeutic agent, is used broadly herein and includes approved therapeutic agents, those that are under regulatory review, those in ongoing clinical trials, and those generally known to be effective. The therapeutic level for effectiveness of the therapeutic agent should be known. In general, the therapeutic agent will be known to be effective to treat a specific condition and its dosage level for treating that specific condition will also be known. In one embodiment, the TDC can be made using the same structure of the therapeutic drug as the carrier of the cytotoxic agents. In another embodiment, the TDC can be produced by conjugating the cytotoxic agent to a molecule that has no therapeutic activity but is antigenically cross-reactive with the therapeutic agents.

The inventors have determined a short course of prophylactic treatment with TDC at doses significantly lower than the therapeutic doses of the therapeutic drug induced long-lasting abrogation of ADA when the patient is later exposed to therapeutic doses of the drug. The TDC doses can be a fourth of a therapeutic dosage, a tenth of a therapeutic dosage, a hundredth of a therapeutic dosage or even less. The unexpected efficacy of the low dose TDC in ablating ADA response permits its use in patients with minimal safety concern.

Repeat dosing of the TDC may be envisioned in certain patients at any time that the ADA levels rise causing a drop in serum levels of the therapeutic agent. It is understood that the frequency and timing of any repeat dosing of the TDC to prevent ADA in a patient in need thereof shall depend on the pharmacokinetics of the underlying therapeutic agent.

To abrogate the level of the ADA, the patient can be administered the TDC at a subtherapeutic level on a first day. Optionally, on a subsequent day after administering the first dose of the TDC, such as one day later, two days later, three days later, or similar delay, the patient is administered a second dose of the TDC at a subtherapeutic level. On a subsequent day after administering the second dose of the TDC, the patient is administered the therapeutic agent at a therapeutic level for which the therapeutic agent is known. It should be understood that the number of subtherapeutic administrations can be varied as well as the timing of the subtherapeutic administrations.

In yet another embodiment, if the therapeutic dose of a drug is low enough that higher doses of TDC are well tolerated, then the TDC dose can be equal to or higher than the known therapeutic dose of the therapeutic drug.

The inventors hypothesize that the TDC can be administered at subtherapeutic levels as long as the dose is high enough to allow its binding to B cells via their antigen receptor specific to the therapeutic agent. Upon binding to the B cells, the cytotoxic agent will specifically destroy the B cells that have the potential to be activated to make ADA in response to future exposure of the therapeutic agents. The TDC should be administered at least once prior to administering the therapeutic agent at a therapeutic level. In some embodiments, the TDC should be administered additional times prior to administering the therapeutic agent at a therapeutic level.

The inventors have tested the hypothesis of subtherapeutic dosing by applying the hypothesis to a proprietary antibody against CD24, called herein ONC781. As set out in the examples below, the inventors have been able to abrogate the levels of the ADA against ONC781 by administering a therapeutic agent conjugated to a cytotoxic agent at subtherapeutic levels of the proprietary antibody ONC781. The inventors have demonstrated that prophylactic treatment with a suitable TDC prevented ADA production, increased drug accumulation, abrogated anaphylaxis and increased therapeutic activity of the therapeutic drug.

### Anti-CD24 Antibody:

The anti-CD24 antibody may specifically target a cancer-specific glycoform of CD24. Specifically, the anti-CD24 antibody or antigen binding fragment thereof may bind to a glycan-shielded epitope that is exposed on cancer cells but not on non-cancerous cells. The anti-CD24 antibody or antigen binding fragment thereof may bind to a CD24 peptide comprising the amino acid sequence SNSGLAPN (SEQ ID NO: 27). The anti-CD24 antibody may be as described in WO2019222082, the contents of which are incorporated herein by reference.

In one embodiment, the anti-CD24 antibody comprises a heavy chain variable region and a light chain variable region of 3B6. The heavy chain variable region may comprise the following sequence.

(SEQ ID NO: 1)
EVKFEESGGGLVQPGGSIKLSCAASGVTFSEAWMDWVRQSPEKGLEWVA
EIRDKTKNYVTYYAESVKGRFTISRDDSKSRVYLQMNNLRTEDTGIYYC
TGAMDYWGQGTSVTVSS

The light chain variable region may comprise the following sequence.

(SEQ ID NO: 2)
DIVMTQTPLSLSVTIGQPASISCKSSQSLLYSNGKTYLNWLQQRPGQSP
KRLIYQVSKLDPGIPDRFSGSGSETDFTLKISRVEAEDLGIYYCLQGTS
YPWTFGGGTKLEIKR

The anti-CD24 antibody or antigen binding fragment thereof may comprise a heavy chain variable region and a light chain variable region of an affinity-matured version of 3B6. The anti-CD24 antibody may comprise a heavy chain variable region comprising one of the following sequences.

(SEQ ID NO: 3)
EVKFEESGGGLVQPGGSIKLSCAASGVAFSGAWMDWVRQSPEKGLEWVA
EIRDKTKNYVTYYAESVKGRFTISRDDSKSRVYLQMNNLRTEDTGVYYC
TGAMDYWGQGTSVTVSS
(SEQ ID NO: 4)
EVKFEESGGGLVQPGGSIKLSCAASGVTFSEAWMDWVRQSPEKGLEWVA
EIRDKSTNYVTYYAESVKGRFTISRDDSKSRVYLQMNNLRTEDTGIYYC
TGAMDYWGQGTSVTVSS
(SEQ ID NO: 5)
EVKFEESGGGLVQPGGSIKLSCAASGVTFSEAWMDWVRQSPEKGLEWVA
EIRDNTTNYVTYYAESVKGRFTISRDDSKSRVYLQMNNLRTEDTGIYYC
TGAMDYWGQGTSVTVSS
(SEQ ID NO: 6)
EVKFEESGGGLVQPGGSIKLSCAASGVTFSEAWMDWVRQSPEKGLEWVA
EIRDKPNSYVTYYAESVKGRFTISRDDSKSRVYLQMNNLRTEDTGIYYC
TGAMDYWGQGTSVTVSS
(SEQ ID NO: 7)
EVKFEESGGGLVQPGGSIKLSCAASGVPFSGAWMDWVRQSPEKGLEWVA
EIRDKTKNYVTYYAESVKGRFTISRDDSKSRVYLQMNNLRTEDTGIYYC
TGAMDYWGQGTSVTVSS
(SEQ ID NO: 8)
EVKFEESGGGLVQPGGSIKLSCAASGVTFSEAWMDWVRQSPEKGLEWVA
EIRDKTKNYVTYYAESVKGRFTISRDDSKGRVYLQMNNLRTEDTGIYYC
TGAMDYWGQGTSVTVSS
(SEQ ID NO: 9)
EVKFEESGGGLVQPGGSIKLSCAASGVTFSEAWMDWVRQTPEKGLEWVA
EIRDRETKYVTYYAESVKGRFTISRDDSKSRVYLQMNNLRTEDTGIYYC
TGAMDYWGQGTSVTVSS
(SEQ ID NO: 10)
EVKFEESGGGLVQPGGSIKLSCAASGVTFSEAWMDWVRQSPEKGLEWVA
EIRDKQNEYVTYYAESVKGRFTISRDDSKSRVYLQMNNLRTEDTGIYYC
TGAMDYWGQGTSVTVSS

The anti-CD24 antibody may comprise a light chain variable region comprising one of the following sequences.

(SEQ ID NO: 11)
DIVMTQTPLSLSVTIGQPASISCKSSQSLLYSNGKTYLNWLQQRPGQSP
LDPGTPDRFSGSGSETDFTLKISRVEAEDLGIYYCMQGTSTPWTFGGGT
KKRLIYQVSKLEIKR
(SEQ ID NO: 12)
DIVMTQTPLSLSVTIGQPASISCKSSQSLLYSNGKTYLNWLQQRPGQSP
KRLIYQVSKLDPGIPDRFSGSGSETDFTLKISRVEAEDLGIYYCMQGAS
LPWTFGGGTKLEIKR
(SEQ ID NO: 13)
DIVMTQTPLSLSVTIGQPASISCKSSQSLLYSNGKTYLNWLQQRPGQSP
KRLIYQVSKLDPGTPDRFSGSGSETDFTLKISRVEAEDLGIYYCMQGAS
VPWTFGGGTKLEIKR
(SEQ ID NO: 14)
DIVMTQTPLSLSVTIGQPASISCKSSQSLLYSNGKTYLNWLQQRPGQSP
KRLIYQVSKLDPGIPDRFSGSGSETDFTLKISRVEAEDLGIYYCMQGTY
LPWTFGGGTKLEIKR
(SEQ ID NO: 15)
DIVMTQTPLSLSVTIGQPASISCKSSQSLLYSNGKTYLNWLQQRPGQSP
KRLIYQVSKLDPGIPDRFSGSGSETDFTLKISRVEAEDLGIYYCMQGTS
LPWTFGGGTKLEIKR
(SEQ ID NO: 16)
DIVMTQTPLSLSVTIGQPASISCKSSQSLLYSNGKTYLNWLQQRPGQSP
KRLIYQVSKLDPGIPDRFSGSGSETDFTLKISRVEAEDLGIYYCMQGSS
LPWTFGGGTKLEIKR

In one example, the anti-CD24 antibody or antigen binding fragment thereof comprises the heavy and light chain variable regions of PP6373, which may comprise the heavy chain variable region comprising the sequence set forth in SEQ ID NO: 6 and the light chain variable region comprising the sequence set forth in SEQ ID NO: 16.

The anti-CD24 antibody or antigen binding fragment thereof may be a humanized version of PP6373 and may comprise a heavy chain variable region comprising one of the following sequences.

(H1)
(SEQ ID NO: 17)
EVQFVESGGGLVQPGGSLKLSCAASGVTFSEAWMDWVRQASGKGLEWVGE
IRDKPNSYVTYYAESVKGRFTISRDDSKNTAYLQMNSLKTEDTAVYYCTG
AMDYWGQGTLVTVSS
(H2)
(SEQ ID NO: 18)
EVQFVESGGGLVQPGGSLKLSCAASGVTFSEAWMDWVRQASGKGLEWVAE
IRDKPNSYVTYYAESVKGRFTISRDDSKSTAYLQMNSLKTEDTAVYYCTG
AMDYWGQGTLVTVSS
(H3)
(SEQ ID NO: 19)
EVQFVESGGGLVQPGGSLKLSCAASGVTFSEAWMDWVRQASGKGLEWVAE
IRDKPNSYVTYYAESVKGRFTISRDDSKSTAYLQMNSLKTEDTAIYYCTG
AMDYWGQGTLVTVSS
(H4)
(SEQ ID NO: 20)
EVQFVESGGGLVQPGGSLKLSCAASGVTFSEAWMDWVRQASGKGLEWVAE
IRDKPNSYVTYYAESVKGRFTISRDDSKSTVYLQMNSLKTEDTAIYYCTG
AMDYWGQGTLVTVSS

The humanized anti-CD24 antibody may comprise a light chain variable region comprising one of the following sequences.

(L1)
(SEQ ID NO: 21)
DIVMTQTPLSLSVTPGQPASISCKSSQSLLYSNGKTYLNWLLQKPGQSPQ
RLIYQVSKLDPGVPDRFSGSGSETDFTLKISRVEAEDVGVYYCMQGSSLP
WTFGGGTKVEIKR
(L2)
(SEQ ID NO: 22)
DIVMTQTPLSLSVTIGQPASISCKSSQSLLYSNGKTYLNWLLQKPGQSPQ
RLIYQVSKLDPGVPDRFSGSGSETDFTLKISRVEAEDVGVYYCMQGSSLP
WTFGGGTKVEIKR
(L3)
(SEQ ID NO: 23)
DIVMTQTPLSLSVTIGQPASISCKSSQSLLYSNGKTYLNWLLQKPGQSPK
RLIYQVSKLDPGIPDRFSGSGSETDFTLKISRVEAEDVGIYYCMQGSSLP
WTFGGGTKVEIKR
(L4)
(SEQ ID NO: 24)
DIVMTQTPLSLSVTIGQPASISCKSSQSLLYSNGKTYLNWLLQKPGQSPK
RLIYQVSKLDPGVPDRFSGSGSETDFTLKISRVEAEDVGVYYCMQGSSLP
WTFGGGTKVEIKR

In one example, the humanized the anti-CD24 antibody or antigen binding fragment thereof is related to H1L1 and comprises the heavy chain variable region comprising the sequence set forth in SEQ ID NO: 17 and the light chain variable region comprising the sequence set forth in SEQ ID NO: 21.

In another example, the humanized anti-CD24 antibody is related to H3L3 and comprises the heavy chain variable region comprising the sequence set forth in SEQ ID NO: 19 and the light chain variable region comprising the sequence set forth in SEQ ID NO: 23. The ADC comprising this anti-CD24 antibody or antigen binding fragment thereof may be referred to as ONC-784.

In a further example, the humanized anti-CD24 antibody is related to H2L3 and comprises the heavy chain variable region comprising the sequence set forth in SEQ ID NO: 18 and the light chain variable region comprising the sequence set forth in SEQ ID NO: 23.

The heavy chain of the anti-CD24 antibody may comprise a heavy chain constant region. The heavy chain constant region may comprise a constant region from an immunoglobulin (Ig), which may be one of IgG, IgM, or IgA. The IgG may be one of IgG1, IgG2, IgG3, or IgG4. In one example, the constant region comprises an Fc region. The Fc region may be of IgG1. The Ig may be human. In one example, the heavy chain constant region comprises the following sequence.

(SEQ ID NO: 25)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPG

The light chain of the anti-CD24 antibody may comprise a light chain constant region. The light chain constant region may comprise the following sequence.

(SEQ ID NO: 26)
TVAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTKS
FNRGEC

### Production of Anti-CD24 Antibodies:

The anti-CD24 antibodies described herein may be prepared using a eukaryotic expression system. The expression system may entail expression from a vector in mammalian cells, such as Chinese Hamster Ovary (CHO) cells. The system may also be a viral vector, such as a replication-defective retroviral vector that may be used to infect eukaryotic cells. The antibodies may also be produced from a stable cell line that expresses the antibody from a vector or a portion of a vector that has been integrated into the cellular genome. The stable cell line may express the antibody from an integrated replication-defective retroviral vector. The expression system may be GPExTM.

The anti-CD24 antibody described herein or antigen binding fragment thereof can be purified using, for example, chromatographic methods such as affinity chromatography, ion exchange chromatography, hydrophobic interaction chromatography, DEAE ion exchange, gel filtration, and hydroxylapatite chromatography. In some embodiments, fusion proteins can be engineered to contain an additional domain containing amino acid sequence that allows the polypeptides to be captured onto an affinity matrix. For example, the antibodies described herein comprising the Fc region of an immunoglobulin domain can be isolated from cell culture supernatant or a cytoplasmic extract using a protein A column. In addition, a tag such as c-myc, hemagglutinin, polyhistidine, or Flag™ (Kodak) can be used to aid polypeptide purification. Such tags can be inserted anywhere within the polypeptide, including at either the carboxyl or amino terminus. Other fusions that can be useful include enzymes that aid in the detection of the polypeptide, such as alkaline phosphatase. Immunoaffinity chromatography also can be used to purify polypeptides.

### Linker:

The linker linking the therapeutic agent to the antibody of an ADC may be short, long, hydrophobic, hydrophilic, flexible or rigid, or may be composed of segments that each independently have one or more of the above-mentioned properties such that the linker may include segments having different properties. The linker may be polyvalent and may covalently link more than one therapeutic agent to a single site on the antibody. The linker may be monovalent and may link a single therapeutic agent to a single site on the antibody.

The linker may link the one or more therapeutic agents to the antibody by forming a covalent linkage to the therapeutic agent at one location and a covalent linkage to antibody at another. The covalent linkage may be formed by reactions between functional groups on the linker and functional groups on one or more cytotoxic agents and the antibody. The linker may be unconjugated, and may comprise a functional group capable of covalently linking the linker to one or more therapeutic agents and a functional group capable of covalently linking the linker to the antibody. The linker may be partially conjugated, and may comprise a functional group that covalently links the linker to the antibody and that is covalently linked to the one or more therapeutic agents, or vice versa. The linker may be covalently linked to both the one or more therapeutic agents and the antibody. The linker may comprise one or more moieties comprising the functional groups on the linker and covalent linkages formed between the linker and the antibody. The linker may be chemically stable to conditions outside the cell, and may be one or more of cleaved, immolated, and otherwise specifically degraded inside the cell.

The linker may not be specifically cleaved, immolated, or degraded inside a cell. The choice of stable versus unstable linker may depend upon the toxicity of the therapeutic agent. For cytotoxic agents that are toxic to normal cells, the ADC may comprise a stable linker. For cytotoxic agents that are selective or targeted and have lower toxicity to normal cells, chemical stability of the linker to the extracellular environment may be less important. A wide variety of linkers useful for linking therapeutic agents to antibodies in the context of ADCs is known in the art. Any of these linkers, as well as other linkers, may be used to link the therapeutic agents to the antibody of the ADCs described herein.

The linker may be polyvalent. Exemplary polyvalent linkers that may be used to link a plurality of therapeutic agents to a single antibody molecule are described, for example, in WO 2009/07345; WO 2010/068795; WO 2010/138719; WO 2011/120053; WO 2011/171020; WO 2013/096901; WO 2014/008375; WO 2014/093379: WO 2014/093394; WO 2014/093640, the content of which are incorporated herein by reference in their entireties. The linker may be a dendritic-type linker. Additional examples of dendritic-type linkers can be found in US 2006/116422; US 2005/271615; de Groot et al (2003) Angew. Chem. Int. Ed. 42:4490-4494; Amir et al (2003) Angew. Chera. Int. Ed. 42:4494-4499; Shamis et al (2004) J. Am. Chem. Soc, 126: 1726-1731; Sun et al (2002) Bioorganic & Medicinal Chemistry Letters 12:2233-2215; Sun et al (2003) Bioorganic & Medicinal Chemistry 1: 1761-1768; King et al (2002) Tetrahedron Letters 43: 1987-1990, each of which is incorporated herein by reference.

The linker may be monovalent. Exemplary monovalent linkers that may be used are described, for example, in Molting, 2013, Antibody-Drug Conjugates, Methods in Molecular Biology 1045:71-100; Kitson et al., 2013.

The linker may be cleavable in vivo. The cleavable linker may include a chemically or enzymatically unstable or degradable linkage. The cleavable linker may rely on processes inside the cell to liberate the therapeutic agent, such as reduction in the cytoplasm, exposure to acidic conditions in the lysosome, or cleavage by specific proteases or other enzymes within the cell. The cleavable linker may incorporate one or more chemical bonds that are either chemically or enzymatically cleavable while the remainder of the linker is non-cleavable. The cleavable linker may be a hydrozone, disulfide, or peptide linker. In one example, the peptide linker is a dipeptide linker. The dipeptide linker may comprise Valine-Cit (VC) and may comprise mc-Val-Cit-PAB (N-[6-(2,5-dihydro-2,5-dioxo-1H-pyrrol-1-yl)-1-oxohexyl]-L-valyl-N5-(aminocarbonyl)-N-[4-(hydroxymethyl)phenyl]-). The dipeptide linker may comprise Val-Ala.

The linker may comprise a chemically labile group such as hydrazone and/or disulfide groups. Linkers comprising chemically labile groups exploit differential properties between the plasma and some cytoplasmic compartments. The intracellular conditions to facilitate therapeutic agent release for hydrazone containing linkers are the acidic environment of endosomes and lysosomes, while the disulfide containing linkers are reduced in the cytosol, which contains high thiol concentrations, e.g., glutathione, in certain embodiments, the plasma stability of a linker comprising a chemically labile group may be increased by introducing steric hindrance using substituents near the chemically labile group. In one example, the cleavable linker comprises CL2A ((6,12,15,18,21,24,27,30,33-Nonaoxa-3,9-diazapentatriacontanamide, 2-(4-aminobutyl)-35-[4-[[[[4-[(2,5-dihydro-2,5-dioxo-1H-pyrrol-1-yl)methyl]cyclohexyl]carbonyl]amino]methyl]-1H-1,2,3-triazol-1-yl]-N-[4-(hydroxymethyl)phenyl]-4,8-dioxo-, (2S)—). In another example, the cleavable linker comprises mc-GGFG ((S)-6-(2,5-Dioxo-2,5-dihydro-1H-pyrrol-1-yl)-N-(2-((2-((1-((2-((4-(hydroxymethyl)phenyl)amino)-2-oxoethyl)amino)-1-oxo-3-phenylpropan-2-yl)amino)-2-oxoethyl)amino)-2-oxoethyl)hexanamide).

Acid-labile groups, such as hydrazone, remain intact during systemic circulation in the blood's neutral pH environment (pH 7.3-7.5) and undergo hydrolysis and release the cytotoxic agent once the ADC is internalized into mildly acidic endosomal (pH 5.0-6.5) and lysosomal (pH 4.5-5.0) compartments of the cell. This pH dependent release mechanism has been associated with nonspecific release of the therapeutic agent. To increase the stability of the hydrazone group of the linker, the linker may be varied by chemical modification, e.g., substitution, allowing tuning to achieve more efficient release of the cytotoxic agent in the lysosome with a minimized loss in circulation. Hydrazone-containing linkers may contain additional cleavage sites, such as additional acid-liable cleavage sites and/or enzymatically labile cleavage sites.

The cleavable linker may comprise a disulfide group. Disulfides are thermodynamically stable at physiological pH and are designed to release the drug upon internalization inside cells, wherein the cytosol provides a significantly more reducing environment compared to the extracellular environment. Scission of disulfide bonds generally requires the presence of a cytoplasmic thiol cefaclor, such as (reduced) glutathione (GSH), such that disulfide-containing linkers are reasonably stable in circulation, selectively releasing the therapeutic agent in the cytosol. The intracellular enzyme protein disulfide isomerase, or similar enzymes capable of cleaving disulfide bonds, may also contribute to the preferential cleavage of disulfide bonds inside cells. GSH is reported to be present in cells in the concentration range of 0.5-10 mM compared with a significantly lower concentration of GSH or cysteine, the most abundant low molecular weight thiol, in circulation at approximately 5 μM. Tumor cells, where irregular blood flow leads to a hypoxic state, result in enhanced activity of reductive enzymes and therefore even higher glutathione concentrations, in certain embodiments, the in vivo stability of a disulfide containing linker may be enhanced by chemical modification of the linker, for example, the use of steric hinderance adjacent to the disulfide bond. In one example, the glutathione-sensitive disulfide linker comprises glutathione-sensitive disulfide linker is SPDB (butanoic acid, 4-(2-pyridinyldithio)-, 2,5-dioxo-1-pyrrolidinyl ester). The disulfide linker may comprise SPP (Nsuccinimidyl-4-(2-pyridyldithio)pentanoate).

The non-cleavable linker may comprise succinimidyl 4-(Nmaleimidomethyl) cyclohexane-1-carboxylate (SMCC).

### Cytotoxic Agent:

The cytotoxic agent may comprise a substance that inhibits or prevents the expression activity of cells, function of cells and/or causes destruction of cells. The cytotoxic agent may comprise one or more radioactive isotopes, chemotherapeutic agents, immune modulators, and toxins such as small molecule toxins or protein-based toxins of bacterial, fungal, plant or animal origin, including fragments and/or variants thereof. Examples of cytotoxic agents include, but are not limited to, auristatins, auromycins, maytansinoids, topoisomerase I or II inhibitors, ricin, ricin A chain, combrestatin, duocarmycins, dolastatins, doxorubicin, daunorubicin, taxols, cisplatin, ccl065, ethidium bromide, mitomycin, etoposide, tenoposide, vincristine, vinblastine, colchicine, dihydroxy anthracin dione, actinomycin, diphtheria toxin, Pseudomonas exotoxin (PE) A, PE40, abrin, abrin A chain, modeccin A chain, alpha-sarcin, gelonin, mitogellin, retstrictocin, phenomycin, enomycin, curicin, crotin, calicheamicin, Sapaonaria officinalis inhibitor, and glucocorticoid and other chemotherapeutic agents, immune modulators, such as cytokines and Toll like receptor agonists, radioisotopes such as At211, Ac225, I131, I125, Y90, Re186, Re188, Sm153, Bi212 or 213, Ra223, Pb212, Tb149, P32 and radioactive isotopes of Lu including Lu177. The antibody may also be conjugated to an anti-cancer pro-drug activating enzyme capable of converting the pro-drug to its active form.

The term “auristatin” as used herein refers to a family of antimitotic agents. Auristatin derivatives are also included within the definition of the term “auristatin.” Examples of auristatins include, but are not limited to, auristatin E (AE), monomethylauristatin E (MMAE), monomethylauristatin F (MMAF), and synthetic analogs of dolastatin.

The term “maytansinoids” as used herein refers to a family of antimitotic agents, with examples of maytansinoids include, but are not limited to, maytansine and the maytansine thiomethyl analogs S-methyl DM1 (DM1) and S-methyl DM4 (DM4).

### Cytotoxic Agent-Linker Combinations:

The cytotoxic agent may comprise tesirine and the linker may comprise Val-Ala.

The cytotoxic agent and the linker may comprise VC-MMAE. The cytotoxic agent and the linker may comprise the following structure.

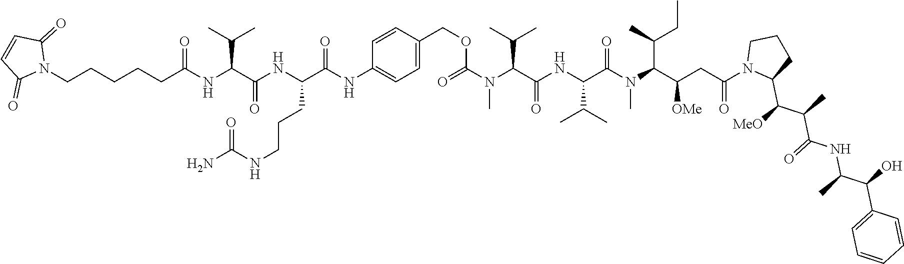 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCCCCN2C(=O)C=CC2=O)C(C)C)cc1)C(C)C]

The cytotoxic agent and the linker may comprise SPDB-DM4. The cytotoxic agent and the linker may comprise the following structure.

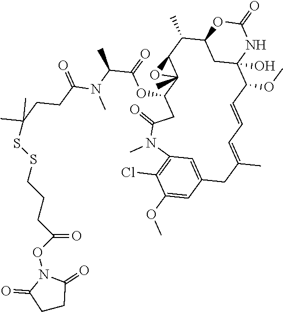 [SMILES: COc1cc2cc(c1Cl)N(C)C(=O)C[C@H](OC(=O)[C@H](C)N(C)C(=O)CCC(C)(C)SSCCCC(=O)ON1C(=O)CCC1=O)[C@]1(C)O[C@H]1[C@H](C)[C@@H]1C[C@@](O)(NC(=O)O1)[C@H](OC)/C=C/C=C(\C)C2]

The cytotoxic agent and the linker may comprise SPP-DM1. The cytotoxic agent and the linker may comprise the following structure.

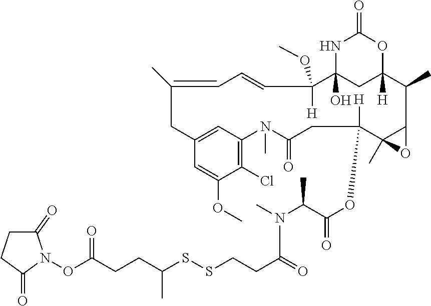

The cytotoxic agent and the linker may comprise SMCC-DM1. The cytotoxic agent and the linker may comprise the following structure.

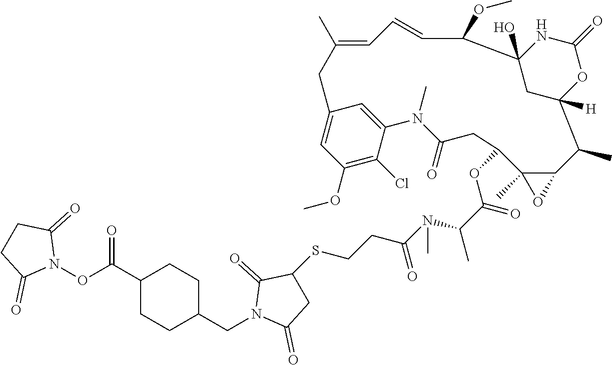

### Method of Conjugation of Antibody to Linker and Cytotoxic Agent:

For VC-MMAE bulk conjugations, mAb A and mAb B (10 mg) in original buffer (PBS, pH 7.0) were pipetted into separate 50 mL tubes, respectively. Next they were reduced by the confirmed optimum TCEP/mAb ratio. Reaction buffer (50 mM PB, pH 7.0) was added to each of the two tubes to make the mAb concentration at 5 mg/nL. The reaction vials were placed in an incubator-shaker at 37° C. with a rotation speed at 60 rpm. After reduction for 2 hours, 10 mM linker-payload in DMA was added to samples to make the drug to mAb ratio at 7.0. DMA solvent was added into each sample to make the organic solvent at 10%. Next, the reactions were incubated at 4° C. for another 1 hour. After 1 hour, the samples were purified via spin desalting column (40 K, 10 mL).

For SPBD-DM4 bulk conjugations, mAb A and mAb B (10 mg) in original buffer (PBS, pH 7.0) were pipetted into separate 50 mL tubes, respectively. Next 10 mM linker-payload in DMA was added to each sample to make the drug to mAb ratio at the confirmed optimum ratio. DMA solvent was added into each sample to make the organic solvent at 15%. Then conjugation buffer (50 mM PB, pH 7.0) was added to each of the two tubes to make the mAb concentration at 5 mg/mL. The reaction vials were placed in an incubator-shaker at 22° C. with a rotation speed of 60 rpm. After reaction for 2.5 hours, all the samples were quenched by 200 mM Succinic acid, pH 2.38 and purified via spin desalting column (40 K, 10 mL).

For SMCC-DM1 conjugation, first a pilot conjugation was performed to determine the optimum drug to mAb ratio and length of conjugation time to achieve desired DAR of 4.0. Bulk conjugation of SMCC-DM1 was performed in phosphate buffer containing 100 mM sodium phosphate and 150 mM sodium chloride, pH 7.25. 10 mg of mAb was pipetted into a 50 ml conical tube followed by addition of 10 mM stock SMCC-DM1 in DMA to make the drug to mAb ratio at the confirmed optimum ratio from the above pilot conjugation. The conjugation reaction was allowed to proceed for the confirmed optimum time at 32° C. Extra DMA was added to obtain a final DMA composition of 10% v/v. After the reaction was complete, unbound SMCC-DM1 was quenched by addition of pH 5.0 succinic acid to give a final succinic acid concentration to be 50 mM and then purified using 40 KD spin desalting column.

The ADCs disclosed herein are listed in Table 1.

TABLE 1
ADC disclosed in the application.

| | | | | Cleavable |
|---|---|---|---|---|
| ADC | | | | or |
| ID | Payload | DAR | Linker | non-cleavable |
| ONC-784-B1 | Tesirine | 2.6 | Val-Ala | Cleavable |
| ONC-784-B5 | DM4 | 3.86 | SPDB | Cleavable |
| ONC-784-B7 | MMAE | 4.04 | VC | Cleavable |
| ONC-784-B8 | DM1 | 3.8 | SMCC | Non-cleavable |
| ONC-784-B10 | DM1 | 3.6 | SPP | Cleavable |

### Example 1: Impact of Different TDCs on Immunogenicity, Drug Accumulation and Therapeutic Activity

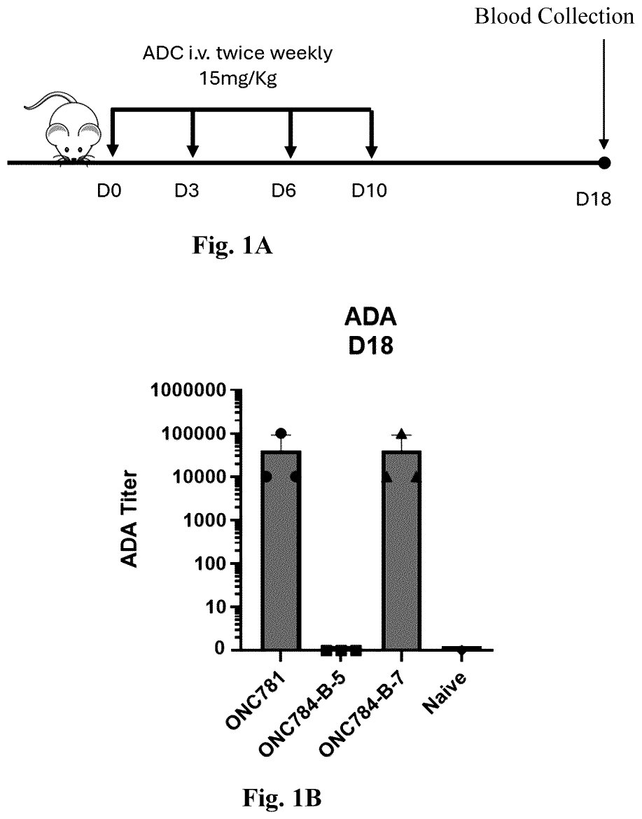

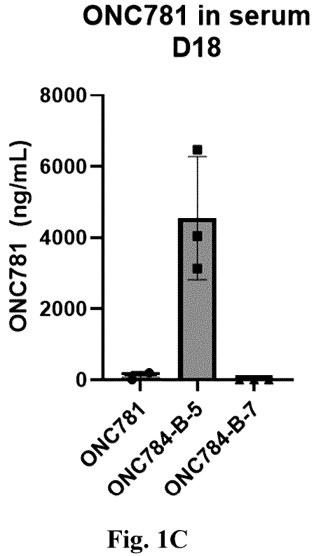

We first compared derivatives of humanized anti-human CD24 mAb ONC-781 (naked antibody), ONC-784-B5 and ONC-784-B7 ADCs for their immunogenicity in mice with a knock in of human CD24 gene, called CD24^{h/h} mice. As shown in FIGS. 1A-B, after 4 doses at 15 mg/kg, twice a week (FIG. 1A), high ADA levels were detected when mice received human IgG such as ONC-781 and ONC-784-B7 (FIG. 1B). Surprisingly, no ADA was detected after repeated dosing of ONC-784-B5. (FIG. 1B). High levels of ONC781 in serum were found for the ONC784-B-5 group while no or very low levels were detected for the ONC781 and ONC784-B-7 groups (FIG. 1C). This supports the hypothesis that because the ADA was eliminated for ONC784-B-5, the ONC781 would not be subject to elimination by the ADA.

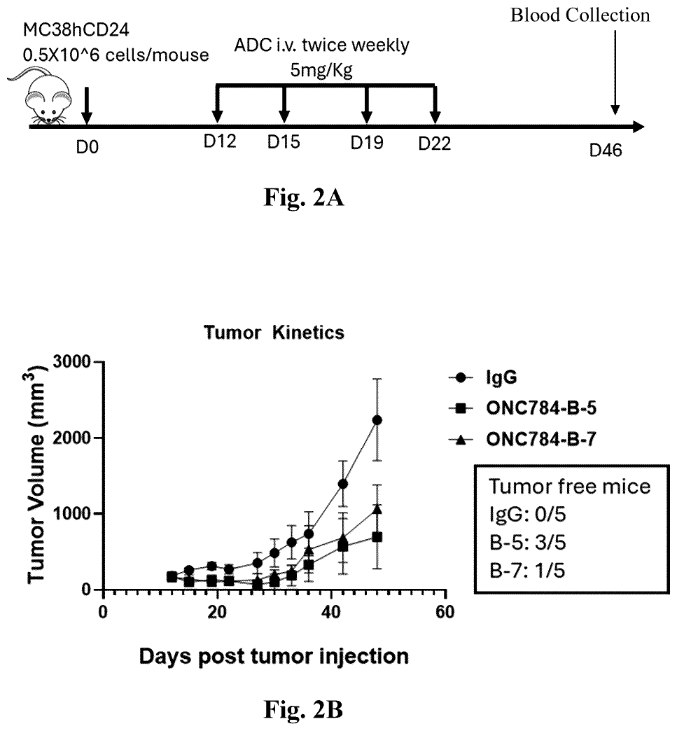

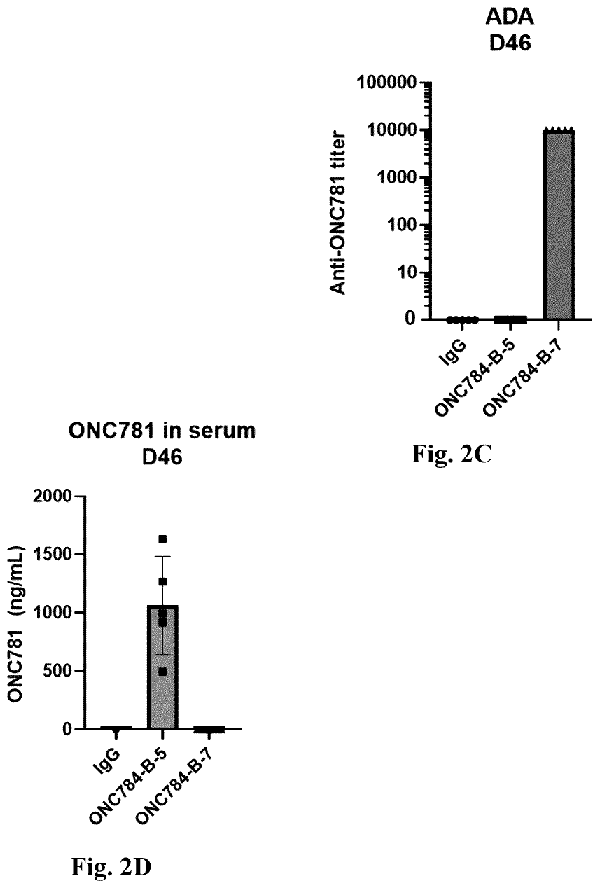

To test whether the difference in immunogenicity affects drug accumulation and anti-tumor activity in syngeneic mice, we transplanted the human CD24-transfected MC38 (MC38-CD24) CD24^{h/h} mice and evaluated therapeutic activity of ONC-784-B5 and ONC-784-B7 (FIG. 2A). As shown in FIG. 2B, while both ADCs had anti-tumor activity, ONC-784-B5 is more active as 3/5 mice completely rejected the tumors. Better anti-tumor activity correlated negatively with ADA levels, while ADA production correlated negatively with the level of drug accumulation. These data demonstrate the significance of preventing ADA production.

### Example 2: Low Dose of ADC Induced Long-Lasting Tolerance to High Dose of Therapeutic Antibody

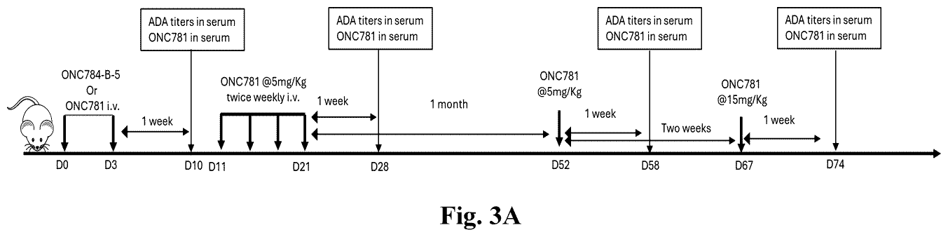

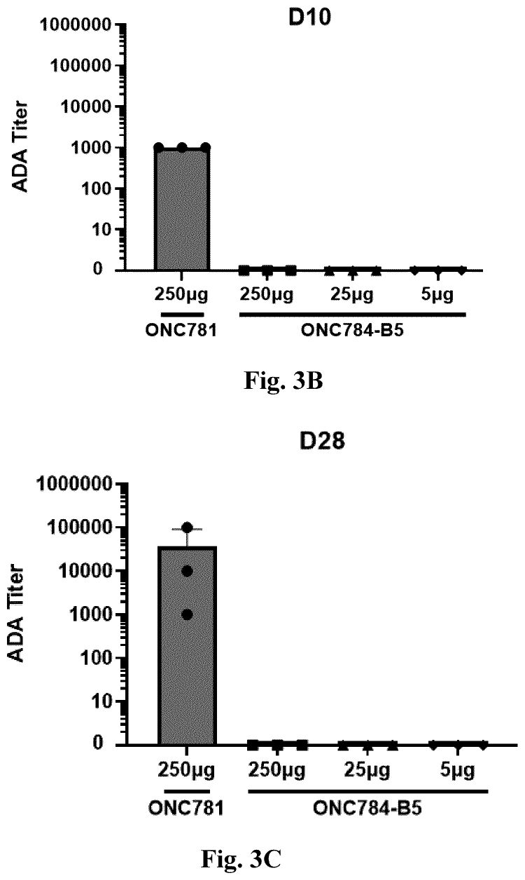

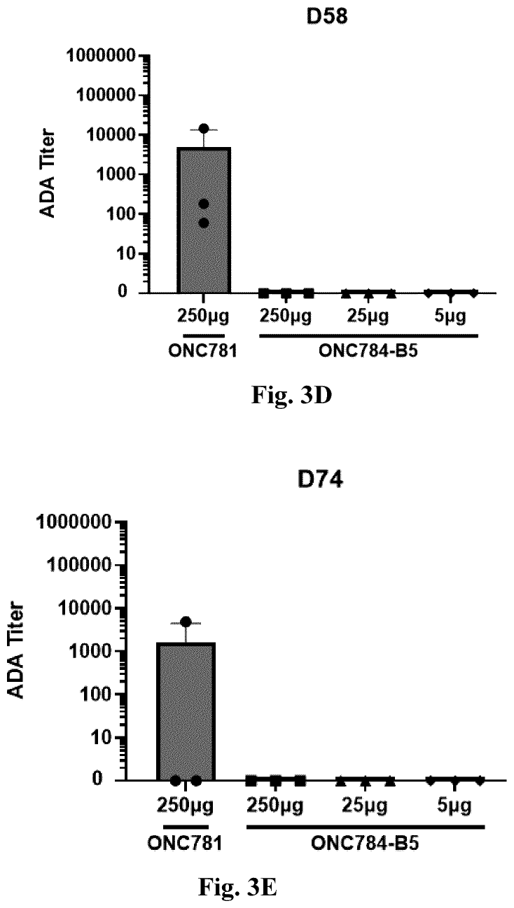

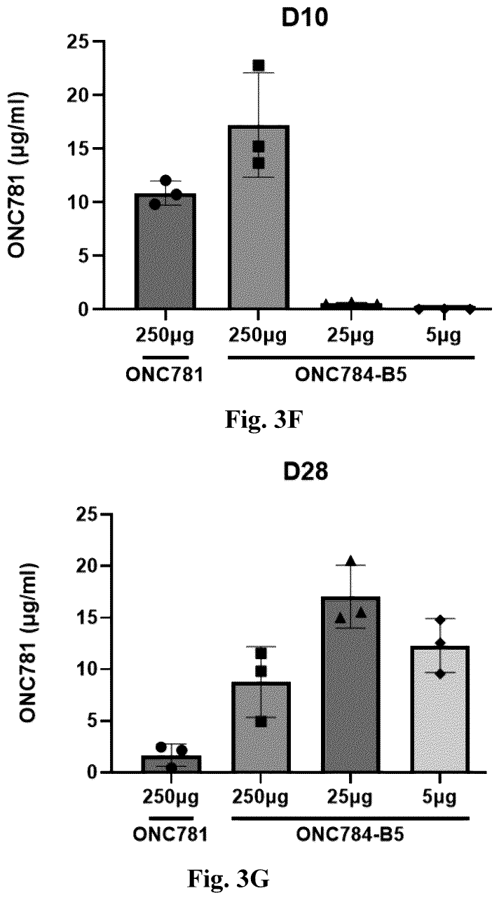

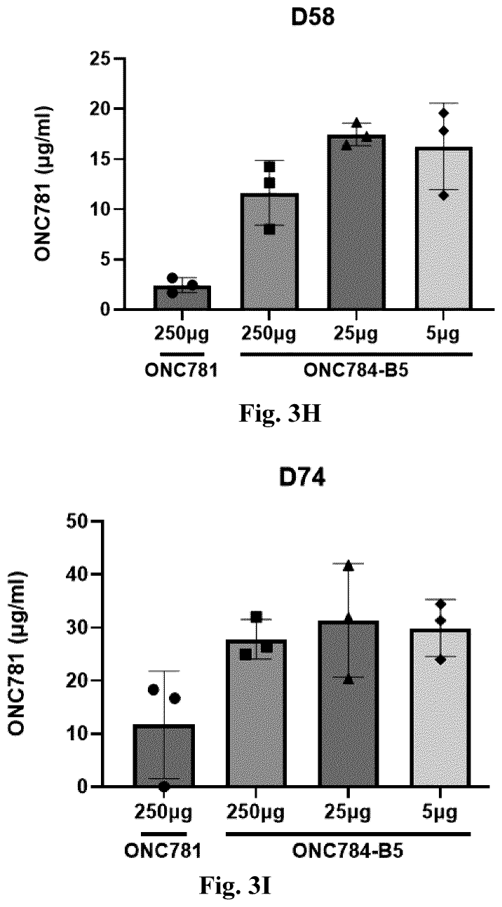

In order to avoid toxicity associated with a TDC, it is desirable if a low dose of TDC would be sufficient to induce lasting tolerance to therapeutic proteins. To explore this possibility, we pretreated mice with grading doses of ONC-784-B5, namely 250 μg/mouse, 25 μg/mouse, and 5 μg/mouse, on Day 1 and Day 3. The mice then were challenged with a therapeutic dose (5 mg/kg) or super-high dose (15 mg/kg) of therapeutic protein to evaluate the strength and duration of immune tolerance to ONC-781 (FIG. 3A).

As shown in FIG. 3B, at all doses tested ONC-784-B5 induced no ADA after initial dosing, in contrast to ONC-781, which suggested that adding cytotoxic drugs to ONC-781 prevented ADA induction. More importantly, at all dose levels, no ADA was induced by repeated dosing of therapeutic and super therapeutic challenges with the therapeutic protein, ONC-781, throughout the 2.5 months of observation period (FIGS. 3B-E). Furthermore, pretreatment of ONC-784-B5 at all doses increased accumulation of therapeutic proteins (FIGS. 3F-I). Taken together, the data presented in this example demonstrated that at sub-therapeutic or super-therapeutic doses, TDC can induce long-lasting and essentially complete tolerance to therapeutic protein at both therapeutic and super therapeutic doses. These data enabled methods to use a TDC to prevent ADA production and enhance future exposure to therapeutic proteins. In one embodiment, the TDC can be used at sub-therapeutic doses, which is at least 20-fold lower than that of therapeutic doses of the therapeutic protein. The sub-therapeutic dosing is desirable as it may help to reduce safety concerns of therapeutic drug. In another embodiment, the TDC can be used at doses equal or higher than therapeutic doses if the TDC is well tolerated at these doses.

### Example 3: Use of Sub-Therapeutic Doses of TDC to Prevent ADA and Lethal Anaphylaxis of Another TDC with Different Payload and Linker

The fact that a TDC such as ONC-784-B7 can induce ADA (FIGS. 1A-C and 2A-D) raised two important questions. First, does the ADA to a TDC increase the risk of anaphylaxis in repeated dosing? Second, can ADA and anaphylaxis be prevented by a tolerance-inducing TDC such as ONC-784-B5? We performed an extensive study to address both issues by comparing the risk of fatal anaphylaxis in ADA inducing and non-inducing TDCs and then testing if pretreatment with subtherapeutic doses of ONC-784-B5 prevented ADA and anaphylaxis associated with ONC-785-B7.

First, we compared the risk of fatal anaphylaxis in ADA inducing and non-inducing TDCs in the CD24^{h/h} mice. As shown in Table 2, at various doses tested, ONC-784-B5 induced no detectable ADA and caused no fatal anaphylaxis after 4 weekly dosings. In contrast, ONC-784-B7 induced ADA after two doses, and most mice (7/9 female) that received 3 doses died of anaphylaxis on the third dosing. The high incidence of anaphylaxis prompted us to halt dosing to the male mice.

Four to 8 weeks old CD24^{h/h} mice received intravenous dosing of ONC-781, ONC784-B5, or ONC784-B7 at 6 mg/kg, 12 mg/kg and 24 mg/kg dose Q1W for a total of 4 planned injections. Control mice received PBS. After administration, peripheral blood was collected every two weeks to measure anti-ONC-781 ADA levels. Each experimental group had 3 female and 3 male mice, totaling 6 mice per group. Male mice were dosed one day behind female mice. Due to significant hypersensitivity reactions in the ONC-784-B7-dosed female mice at the third dosing, dosing was halted for males after receiving 2 doses. One male mouse in the 12 mg/kg ONC-784-B7 dose group did not yield sufficient peripheral blood during the first collection. Serum was diluted 1:500 with PBS, and the anti-ONC-781 ADA levels were measured using the ELISA. Positive ADA defined as OD reach 2× of the average of the signal from the blank control mouse serum.

TABLE 2
Comparison between ONC-784-B5 and ONC-
784-B7 for ADA and fatal anaphylaxis.

| | | ADA Incidence | |
|---|---|---|---|
| | | at one | Fatal anaphylaxis |
| | | week after | incidence at |
| Drug | Dose | 2^{nd} dose | final dose |
| ONC-781 | | | |
| | 6 mg/kg Q1Wx4 | 6/6 | 4/6 |
| | 12 mg/kg Q1Wx4 | 5/6 | 3/6 |
| | 24 mg/kg Q1Wx4 | 3/6 | 4/6 |
| ONC-784-B3 | | | |
| | 6 mg/kg Q1Wx4 | 0/6 | 0/6 |
| | 12 mg/kg Q1Wx4 | 0/6 | 0/6 |
| | 24 mg/kg Q1Wx4 | 0/6 | 0/6 |
| ONC-784-B7 Dar4.0 | | | |
| | 6 mg/kg Q1Wx2(M) or 3(F) | 5/6 | 2/6 |
| | 12 mg/kg Q1Wx2(M) or 3(F) | 5/5 | 3/6 |
| | 24 mg/kg Q1Wx2(M) or 3(F) | 6/6 | 3/6 |

Second, we tested if pretreatment with subtherapeutic doses of ONC-784-B5 prevented ADA and anaphylaxis associated with the ONC-784-B7 using a dosing regimen known to induce anaphylaxis associated with ADA production. As shown in Table 3, pre-treatment with ONC-784-B5 prevented all but 1 out of 75 ONC-784-B7 treated mice from developing any ADA, with no mice developing anaphylaxis. These data demonstrated the power of sub-therapeutic dosing of TDCs such as ONC-784-B5 in the prophylaxis of ADA and anaphylaxis for subsequent exposures to other TDCs.

Three to 8 weeks old CD24^{h/h} mice received 5 μg of ONC-784-B5 via tail vein injection every three days for a total of 2 injections. After one-week, different preparation of ONC-784-B7 with slightly different amounts of payloads were administered at 15 mg/kg dose intravenously every three days for a total of four injections. After the completion of ONC-784-B7 administration, anti-ONC-781 ADA levels in peripheral blood of mice were measured at 1 week and 1-month post-treatment. Each experimental group had 13 female and 12 male mice, totaling 25 mice per group. Serum was diluted 1:500 with PBS, and the anti-ONC-781 ADA levels were measured using the ELISA method. The OD value was read at 450 nm. Positive ADA defined as OD reach 2× of the average of the signal from the blank control mouse serum.

For comparison, 4-8 weeks CD24^{h/h} mice were either pretreated with 10 mg/kg anti-CD20 per mouse or without pretreatment. Flow cytometry using anti-mouse CD19 antibodies was used to assess B cell depletion. Based on the extent of B cell depletion, mice were divided into two groups, each consisting of 3 female and 3 male mice, totaling 6 mice per group. Each group of 6 mice received ONC-784-B7 at 15 mg/kg dose intravenously every three days for a total of four injections.

TABLE 3
Prophylaxis of ADA and fatal anaphylaxis

| Prophylactic TDC | | Time after | | | Fatal |
|---|---|---|---|---|---|
| (ONC-784-B5 at | Therapeutic | dosing (15 | | ADA | anaphylaxis |
| 5 μg/mouse Q3dx2) | drug | mg/kg Q3dx4) | | Incidence* | incidence |
| | ONC-784-B7 DAR3.5 | | | | |
| Yes | | 1 | week | 0/25 | 0/25 |
| Yes | | 1 | month | 1/25 | 0/25 |
| | ONC-784-B7 DAR3.8 | | | | |
| Yes | | 1 | week | 0/25 | 0/25 |
| Yes | | 1 | month | 0/25 | 0/25 |
| | ONC-784-B7 DAR4.1 | | | | |
| Yes | | 1 | week | 0/25 | 0/25 |
| Yes | | 1 | month | 0/25 | 0/25 |

| | | | | |
|---|---|---|---|---|
| | ONC-784-B7 DAR4.3 | | | |
| No | Without B cell-depletion | At 3^{rd} dose | NA | 2/6 |
| | | At 4^{th} dose | NA | 4/4 |
| No | With B cell-depletion | At 3^{rd} dose | NA | 0/6 |
| | | At 4^{th} dose | NA | 0/6 |

### Example 4: A Platform to Identify Therapeutic Drug Conjugates Capable of Preventing ADA Production

Given the diversity of the therapeutics that require effective methods to overcome ADA barrier, we developed a platform to screen for TDCs that can be used to prevent the development of ADA.

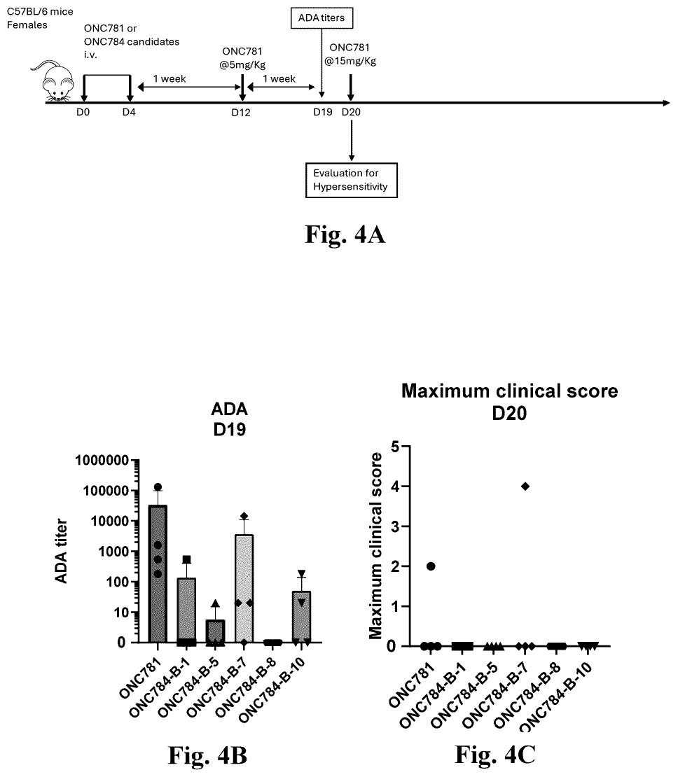

As an example, we evaluated the ADCs in Table 1 for their ability to induce tolerance to future challenges with a therapeutic protein, using ONC-781 as an example. The treatment schedule is shown in FIG. 4A, and the results are shown in FIG. 4B. Briefly, ONC784 ADC candidates were given two pretreatments one week prior to administration of the therapeutic agent ONC781 at 5 μg per intravenous injection per mouse. As a negative control, ONC781 was also given as two injections of 5 μg per injection. ADA was measured one week after challenge with ONC-781 (therapeutic protein) at therapeutic dose. As shown in FIG. 4B, compared with mice dosed with the therapeutic protein control, there were about 200-20,000 fold reduction in mean ADA titer in mice pre-treated with ONC-784-B1 (200-fold), ONC-784-B5 (4,000 fold), ONC-784-B8 (>20,000 fold) and ONC-784-B10 (400-fold). The apparent ADA titer reduction in the ONC-784-B7 group is difficult to ascertain due to large intragroup variation. The successful candidates include different payload and/or linker, suggesting that as a class ADC known in the art can be effective as TDC for the purpose of ADA prevention. These data demonstrate that the method disclosed herein allows rapid identification of TDCs that can be used to prevent ADA production.

As illustrated in FIG. 4C, the maximum clinical score for each ADC group in this study was evaluated at Day 20. Table 4 provides a summary of the data of anaphylaxis response. The maximum clinical score was evaluated with the following protocol. After the second injection of ONC781, mice were observed for anaphylaxis response. Every 10 minutes up to 60 minutes post-ONC781 administration, mice were evaluated for clinical score using a clinical scoring scheme adapted from Flaherty (2012) as described below. The maximum score indicates the highest clinical score observed during the evaluation window.
0—Animal appears to have normal clinical observations and is bright, alert, and responsive.
1—Animal is in a hunched position, moving less frequently and slower but will move if stimulated.
2—Animal is in a hunched position with scruffy fur. The animal is not moving and will not respond to stimulation.
3—Animal is recumbent, cold to touch, and is having difficulty breathing.
4—Animal is dead.
Reference: Flaherty, M. M., MacLachlan, T. K., Troutt, M., Magee, T., Tuaillon, N., Johnson, & Andrews, L. (2012). Nonclinical evaluation of GMA161—an antihuman CD16 (FcγRIII) monoclonal antibody for treatment of autoimmune disorders in CD16 transgenic mice. Toxicological Sciences, 125(1), 299-309.

TABLE 4
Signs of Anaphylaxis Response

| | | Number |
|---|---|---|
| | Number of mice with | of |
| Group | signs of Anaphylaxis Response | Deaths |
| ONC-781 | 1/4 | 0/4 |
| ONC-784-B-1 | 0/4 | 0/4 |
| ONC-784-B-5 | 0/4 | 0/4 |
| ONC-784-B-7 | 1/4 | 1/4 |
| ONC-784-B-8 | 0/4 | 0/4 |
| ONC-784-B-10 | 0/4 | 0/4 |

In one embodiment, the platform includes generation of a TDC, comprising of the therapeutic protein conjugated to cytotoxic agents via linkers disclosed herein. In yet another embodiment, the payload can be radioactive isotypes, biologically active toxin, in addition to the small molecule cytotoxic agent disclosed herein. In yet another embodiment, the therapeutic protein maybe modified in ways to avoid unwanted targeting of certain tissues or cells.

In another embodiment, the proteins used for prophylaxis do not necessarily need to have therapeutic activity as only cross-reactive antigenicity is required. Example 5 provides adalimumab and a loss of function variant of adalimumab that maintains cross-reactive antigenicity.

### Example 5: Generation of Adalimumab and Loss of Function Variant and their Corresponding Therapeutic-Drug Conjugates

We first generated a mutant of adalimumab with loss of function, ADM-A-800, in which it lost the ability to bind to human TNF-alpha but that maintains cross-reactive antigenicity. The following antibody drug conjugates (“ADC”) were prepared.

TABLE 4
ADCs

| | Payload/ | | | Cleavable |
|---|---|---|---|---|
| ADC | linker | | | or |
| ID | code | Payload | Linker | Non-cleavable |
| ADM-A-003-B5 | B5 | DM4 | SPDB | Cleavable |
| ADM-A-800-B5 | B5 | DM4 | SPDB | Cleavable |
| ADM-A-800-B8 | B8 | DM1 | SMCC | Non-cleavable |

ADM-A-003-B5 is a conjugate of adalimumab with payload/linker B5. ADM-A-800 is a conjugate of an adalimumab variant, a linker and a cytotoxic agent. The adalimumab variant antigenically mimics adalimumab but has a sufficient number of mutations to abrogate its binding to TNF-alpha while not affecting its ability to interact with anti-drug antibodies.

SPDB-DM4 and SMCC-DM1 have the following structures:

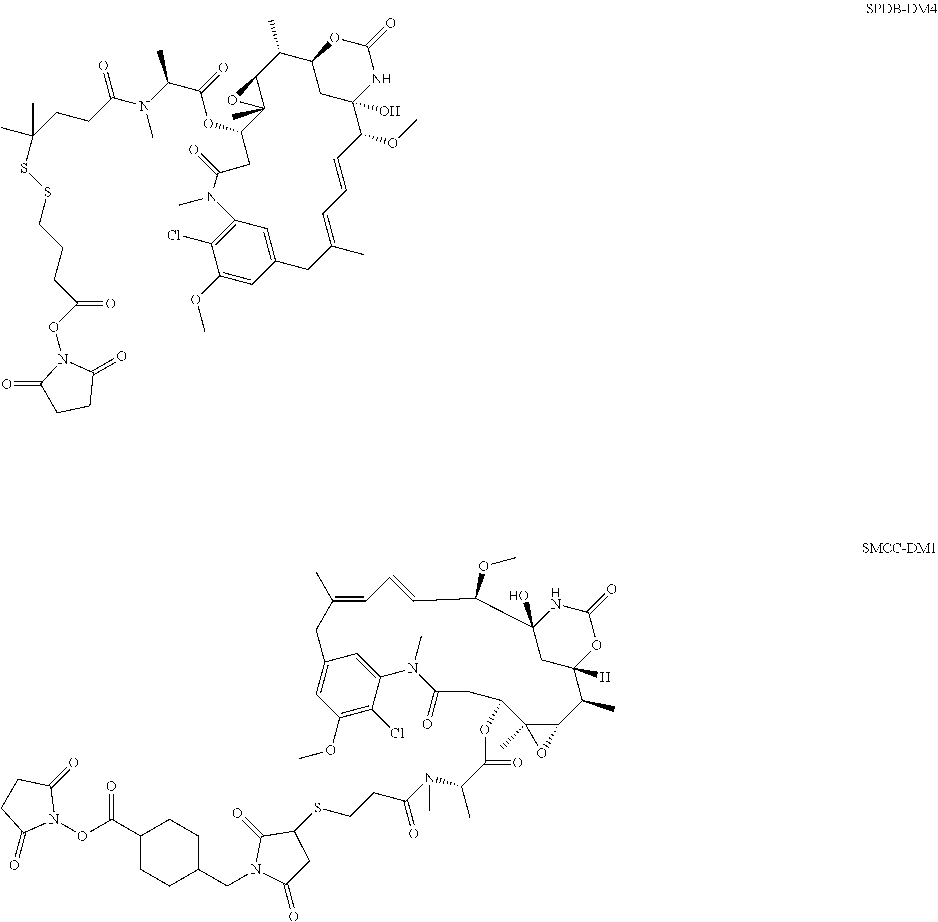 [SMILES: COc1cc2cc(c1Cl)N(C)C(=O)C[C@H](OC(=O)[C@H](C)N(C)C(=O)CCC(C)(C)SSCCCC(=O)ON1C(=O)CCC1=O)[C@]1(C)O[C@H]1[C@H](C)[C@@H]1C[C@@](O)(NC(=O)O1)[C@H](OC)/C=C/C=C(\C)C2 ; COc1cc2cc(c1Cl)N(C)C(=O)C[C@H](OC(=O)[C@H](C)N(C)C(=O)CCSC1CC(=O)N(CC3CCC(C(=O)ON4C(=O)CCC4=O)CC3)C1=O)[C@]1(C)O[C@H]1[C@H](C)[C@@H]1C[C@@](O)(NC(=O)O1)[C@H](OC)/C=C/C=C(\C)C2]

To verify that the mutant had lost its ability to bind to human TNF-alpha, the following ELISA method was followed. Human TNF-alpha was coated at 1 μg/ml onto ELISA 96-well plates overnight at 4° C. The plates were washed three time with PBST and blocked with 3% BSA-PBST for 1 hour at room temperature with shaking. Serial dilution of adalimumab, ADM-A-800, or their corresponding antibody-drug conjugates (ADCs) in 1% BSA-PBST were incubated for two hours with shaking. The plates were washed five times with PBST and incubated with secondary antibody goat anti-human Fc-HRP (Preabsorbed) and incubated for one hour followed by washing the plates five times with PBST. The plates were developed with TMB reagent for 10 minutes and the reaction was stopped with 2N HCl followed by spectrometric reading at OD450.

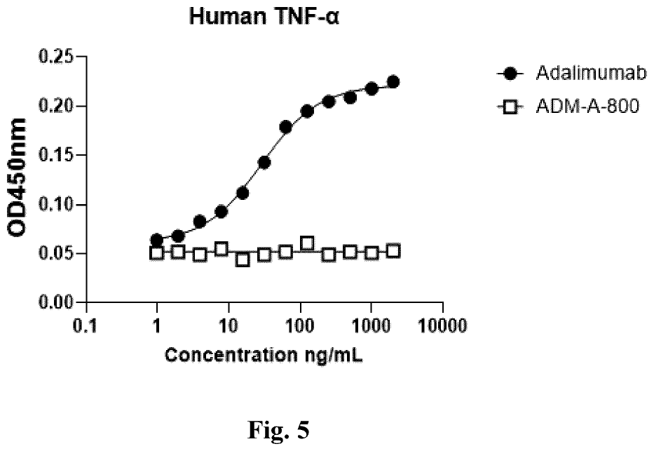

FIG. 5 compares the binding of Adalimumab and ADM-A-800 to human TNF-alpha. The data in FIG. 5 shows that as the concentration of Adalimumab is increased, the amount bound to human TNF-alpha also increases. In contrast, as the concentration of ADM-A-800 is increased, the amount bound to TNF-alpha remains generally constant at a low level. This result confirms the inventor's hypothesis because ADM-A-800 has been designed to lose its function of binding to TNF-alpha.

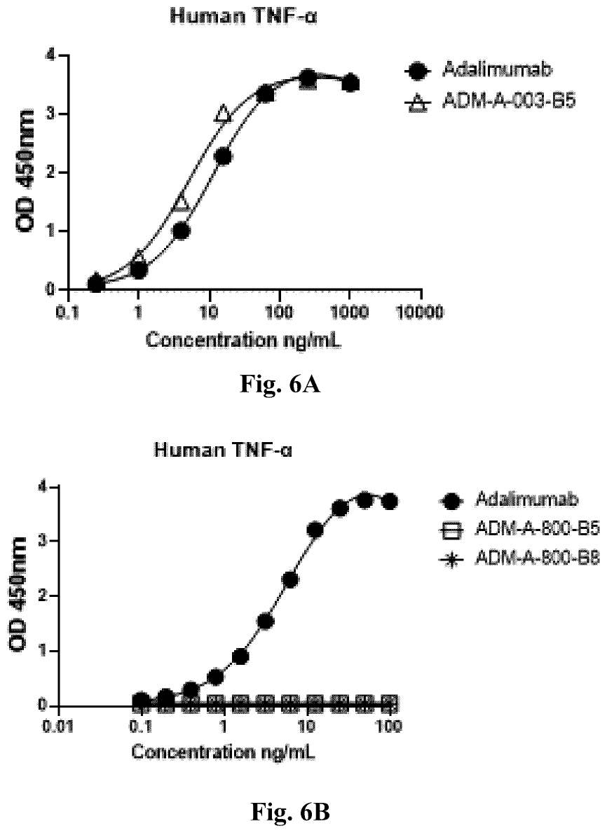

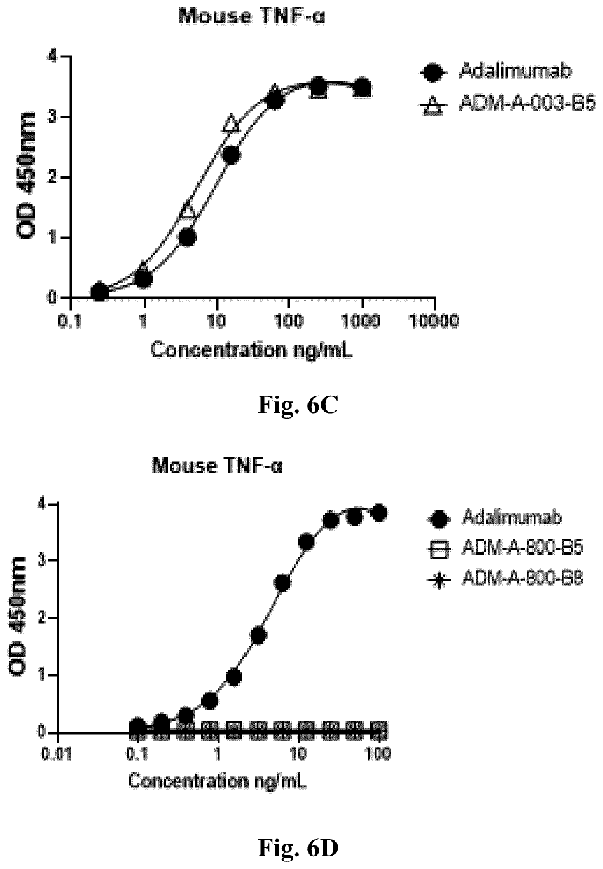

Secondly, we tested the corresponding TDC's for adalimumab and its loss of function variant, ADM-A-800, for their ability to bind to human and mouse TNF-alpha. The ELISA preparation process described above for human TNF-alpha binding was followed except that additional plates were prepared with mouse TNF-alpha. FIGS. 6A and 6C compare the binding capabilities of adalimumab and ADM-A-003-B5 to human and mouse TNF-alpha, respectively. As illustrated in FIGS. 6A and 6C, the Adalimumab and ADM-A-003-B5 bind to a similar extent to each other in both human and mouse TNF-alpha, respectively, with extent of binding increasing with the concentration of the Adalimumab and ADM-A-003-B5. This result confirmed the inventor's hypothesis because ADM-A-003-B5 is designed to include the moiety that binds to TNF-alpha, just as Adalimumab inherently has that binding moiety. FIGS. 6B and 6D compare the binding capabilities of Adalimumab and the loss-of-function variants, ADM-A-800-B5 and ADM-A-800-B8. As illustrated in FIGS. 6B and 6D, Adalimumab binds to human and mouse TNF-alpha while ADM-A-800-B5 and ADM-A-800-B8 appear to have negligible binding. Again, these results confirm the inventor's hypothesis because ADM-A-800-B5 and ADM-A-800-B8 were designed to exclude the moiety that binds to TNF-alpha. In summary, Adalimumab and its corresponding TDC, ADM-A-003-B5, bind similarly to both human and mouse TNF-alpha while adalimumab loss-of-function TDCs, ADM-800-B5 and ADM-800-B8, could not.

1. A method of treatment using a therapeutic agent for a condition which the therapeutic agent is effective, the method comprising:
a. at least one pre-treatment administration to a patient of a therapeutic drug conjugate, wherein the therapeutic drug conjugate is in the form of T-L-C, wherein T comprises the therapeutic agent, L comprises a linker and C comprises a cytotoxic agent; and
b. a first therapeutic administration to a patient of the therapeutic agent,
wherein the first therapeutic administration of the therapeutic agent is at a therapeutic level for treating the condition for which the therapeutic agent is effective and the at least one pre-treatment administration of the therapeutic drug conjugate is at a level that is below the therapeutic level for treating the condition with the therapeutic agent alone.

2. The method of claim 1, wherein the at least one pre-treatment administration of the therapeutic drug conjugate is at a level of the therapeutic agent that is less than one fourth of the therapeutic level of the therapeutic agent alone.

3. The method of claim 1, wherein the at least one pre-treatment administration of the therapeutic drug conjugate is at a level of the therapeutic agent that is less than one tenth of the therapeutic level of the therapeutic agent alone.

4. The method of claim 1, wherein the at least one pre-treatment administration of the therapeutic drug conjugate is at a level of the therapeutic agent that is less than one hundredth of the therapeutic level of the therapeutic agent alone.

5. The method of claim 1, wherein the cytotoxic agent is toxic to a B-cell that produces anti-drug antibodies to the therapeutic.

6. The method of claim 1, wherein the at least one pre-treatment administration and the therapeutic administration are separated by between 1 and 10 days.

7. The method of claim 1, further comprising an additional pre-treatment administration of the therapeutic drug conjugate before the therapeutic administration to the patient of the therapeutic agent.

8. The method of claim 1, further comprising:
c. at least one pre-treatment administration to the patient of the therapeutic drug conjugate T-L-C; and
d. a therapeutic administration to the patient of the therapeutic agent,
wherein the therapeutic administration of the therapeutic agent is at a therapeutic level for treating the condition for which the therapeutic agent is effective and the at least one pre-treatment administration of the therapeutic drug conjugate is at a level that is below the therapeutic level for treating the condition with the therapeutic agent alone, and wherein step (c) occurs after step (b).

9. A method of modulating the levels of an anti-drug antibody, the method comprising administering the T-L-C of claim 1.

10. The method of claim 1, wherein the cytotoxic agent comprises auristatins, auromycins, maytansinoids, topoisomerase I or II inhibitors, ricin, ricin A-chain, combrestatin, duocarmycins, dolastatins, doxorubicin, daunorubicin, taxols, cisplatin, ccl065, ethidium bromide, mitomycin, etoposide, tenoposide, vincristine, vinblastine, colchicine, dihydroxy anthracin dione, actinomycin, diphtheria toxin, Pseudomonas exotoxin (PE) A, PE40, abrin, abrin A chain, modeccin A chain, alpha-sarcin, gelonin, mitogellin, retstrictocin, phenomycin, enomycin, curicin, crotin, calicheamicin, Sapaonaria officinalis inhibitor, and glucocorticoid and other chemotherapeutic agents, as well as radioisotopes such as At211, Ac225, I131, I125, Y90, Re186, Re188, Sm153, Bi212 or 213, Ra223, Pb212, Tb149, P32 and radioactive isotopes of Lu including Lu177.

11. The method of claim 10, wherein the cytotoxic agent comprises monomethylauristatin E (MMAE).

12. The method of claim 11, wherein the cytotoxic agent comprises mytansoids DM1 or DM4.

13. The method of claim 10, wherein the cytotoxic agent comprises a topoisomerase I inhibitor.

14. The method of claim 13, wherein the topoisomerase I inhibitor comprises SN-38 or Dxd.

15. The method of claim 1, wherein the linker is cleavable or non-cleavable.

16. The method of claim 15, wherein the linker is cleavable.

17. The method of claim 16, wherein the cleavable linker comprises hydrazone, disulfide, or peptide linkers.

18. The method of claim 17, wherein the linker further comprises one or more a disulfide groups.

19. The method of claim 1, wherein the linker forms a covalent linkage to the cytotoxic agent at one location and a covalent linkage to the therapeutic agent at another location.

20. The method of claim 1, where the therapeutic protein is modified in ways to avoid the intended target of the therapeutic protein but remain cross-reactive with the therapeutic protein for antibody recognition.

21. The method of claim 1, where the dose of TDC may be higher than therapeutic protein if such doses are tolerable and required for induction of tolerance to therapeutic protein.

22. The method of claim 1, where the therapeutic may be an antibody, a protein with or without part of antibody structure or viral envelop or capsid proteins that are used for viral or gene therapy vectors.

23. A method of identifying a TDC for the use in preventing ADA production related to a specific therapeutic protein, the method comprising:
a) determining a suitable animal model which is capable of producing ADA upon exposure to a specific therapeutic agent;
b) administering at least one pre-treatment administration of a TDC which targets a specific clonal antigen-specific receptor on a B-cell;
c) administering at least one additional dosing with the therapeutic agent at a therapeutic or super-therapeutic dose; and
d) evaluating the ADA levels in the animal model.

24. The method of claim 23, where the therapeutic agent may be an antibody, a protein with or without part of antibody structure, or viral envelop or capsid proteins that are used for viral or gene therapy vectors.


SEQUENCE LISTING (27 sequences)

SEQ ID NO: 1 - protein, 115 aa, unidentified
EVKFEESGGGLVQPGGSIKLSCAASGVTFSEAWMDWVRQSPEKGLEWVAEIRDKTKNYVTYYAESVKGRFTISRDDSKSRVYLQMNNLRTEDTGIYYCTGAMDYWGQGTSVTVSS

SEQ ID NO: 2 - protein, 113 aa, unidentified
DIVMTQTPLSLSVTIGQPASISCKSSQSLLYSNGKTYLNWLQQRPGQSPKRLIYQVSKLDPGIPDRFSGSGSETDFTLKISRVEAEDLGIYYCLQGTSYPWTFGGGTKLEIKR

SEQ ID NO: 3 - protein, 115 aa, unidentified
EVKFEESGGGLVQPGGSIKLSCAASGVAFSGAWMDWVRQSPEKGLEWVAEIRDKTKNYVTYYAESVKGRFTISRDDSKSRVYLQMNNLRTEDTGVYYCTGAMDYWGQGTSVTVSS

SEQ ID NO: 4 - protein, 115 aa, unidentified
EVKFEESGGGLVQPGGSIKLSCAASGVTFSEAWMDWVRQSPEKGLEWVAEIRDKSTNYVTYYAESVKGRFTISRDDSKSRVYLQMNNLRTEDTGIYYCTGAMDYWGQGTSVTVSS

SEQ ID NO: 5 - protein, 115 aa, unidentified
EVKFEESGGGLVQPGGSIKLSCAASGVTFSEAWMDWVRQSPEKGLEWVAEIRDNTTNYVTYYAESVKGRFTISRDDSKSRVYLQMNNLRTEDTGIYYCTGAMDYWGQGTSVTVSS

SEQ ID NO: 6 - protein, 115 aa, unidentified
EVKFEESGGGLVQPGGSIKLSCAASGVTFSEAWMDWVRQSPEKGLEWVAEIRDKPNSYVTYYAESVKGRFTISRDDSKSRVYLQMNNLRTEDTGIYYCTGAMDYWGQGTSVTVSS

SEQ ID NO: 7 - protein, 115 aa, unidentified
EVKFEESGGGLVQPGGSIKLSCAASGVPFSGAWMDWVRQSPEKGLEWVAEIRDKTKNYVTYYAESVKGRFTISRDDSKSRVYLQMNNLRTEDTGIYYCTGAMDYWGQGTSVTVSS

SEQ ID NO: 8 - protein, 115 aa, unidentified
EVKFEESGGGLVQPGGSIKLSCAASGVTFSEAWMDWVRQSPEKGLEWVAEIRDKTKNYVTYYAESVKGRFTISRDDSKGRVYLQMNNLRTEDTGIYYCTGAMDYWGQGTSVTVSS

SEQ ID NO: 9 - protein, 115 aa, unidentified
EVKFEESGGGLVQPGGSIKLSCAASGVTFSEAWMDWVRQTPEKGLEWVAEIRDRETKYVTYYAESVKGRFTISRDDSKSRVYLQMNNLRTEDTGIYYCTGAMDYWGQGTSVTVSS

SEQ ID NO: 10 - protein, 115 aa, unidentified
EVKFEESGGGLVQPGGSIKLSCAASGVTFSEAWMDWVRQSPEKGLEWVAEIRDKQNEYVTYYAESVKGRFTISRDDSKSRVYLQMNNLRTEDTGIYYCTGAMDYWGQGTSVTVSS

SEQ ID NO: 11 - protein, 113 aa, unidentified
DIVMTQTPLSLSVTIGQPASISCKSSQSLLYSNGKTYLNWLQQRPGQSPKRLIYQVSKLDPGTPDRFSGSGSETDFTLKISRVEAEDLGIYYCMQGTSTPWTFGGGTKLEIKR

SEQ ID NO: 12 - protein, 113 aa, unidentified
DIVMTQTPLSLSVTIGQPASISCKSSQSLLYSNGKTYLNWLQQRPGQSPKRLIYQVSKLDPGIPDRFSGSGSETDFTLKISRVEAEDLGIYYCMQGASLPWTFGGGTKLEIKR

SEQ ID NO: 13 - protein, 113 aa, unidentified
DIVMTQTPLSLSVTIGQPASISCKSSQSLLYSNGKTYLNWLQQRPGQSPKRLIYQVSKLDPGTPDRFSGSGSETDFTLKISRVEAEDLGIYYCMQGASVPWTFGGGTKLEIKR

SEQ ID NO: 14 - protein, 113 aa, unidentified
DIVMTQTPLSLSVTIGQPASISCKSSQSLLYSNGKTYLNWLQQRPGQSPKRLIYQVSKLDPGIPDRFSGSGSETDFTLKISRVEAEDLGIYYCMQGTYLPWTFGGGTKLEIKR

SEQ ID NO: 15 - protein, 113 aa, unidentified
DIVMTQTPLSLSVTIGQPASISCKSSQSLLYSNGKTYLNWLQQRPGQSPKRLIYQVSKLDPGIPDRFSGSGSETDFTLKISRVEAEDLGIYYCMQGTSLPWTFGGGTKLEIKR

SEQ ID NO: 16 - protein, 113 aa, unidentified
DIVMTQTPLSLSVTIGQPASISCKSSQSLLYSNGKTYLNWLQQRPGQSPKRLIYQVSKLDPGIPDRFSGSGSETDFTLKISRVEAEDLGIYYCMQGSSLPWTFGGGTKLEIKR

SEQ ID NO: 17 - protein, 115 aa, unidentified
EVQFVESGGGLVQPGGSLKLSCAASGVTFSEAWMDWVRQASGKGLEWVGEIRDKPNSYVTYYAESVKGRFTISRDDSKNTAYLQMNSLKTEDTAVYYCTGAMDYWGQGTLVTVSS

SEQ ID NO: 18 - protein, 115 aa, unidentified
EVQFVESGGGLVQPGGSLKLSCAASGVTFSEAWMDWVRQASGKGLEWVAEIRDKPNSYVTYYAESVKGRFTISRDDSKSTAYLQMNSLKTEDTAVYYCTGAMDYWGQGTLVTVSS

SEQ ID NO: 19 - protein, 115 aa, unidentified
EVQFVESGGGLVQPGGSLKLSCAASGVTFSEAWMDWVRQASGKGLEWVAEIRDKPNSYVTYYAESVKGRFTISRDDSKSTAYLQMNSLKTEDTAIYYCTGAMDYWGQGTLVTVSS

SEQ ID NO: 20 - protein, 115 aa, unidentified
EVQFVESGGGLVQPGGSLKLSCAASGVTFSEAWMDWVRQASGKGLEWVAEIRDKPNSYVTYYAESVKGRFTISRDDSKSTVYLQMNSLKTEDTAIYYCTGAMDYWGQGTLVTVSS

SEQ ID NO: 21 - protein, 113 aa, unidentified
DIVMTQTPLSLSVTPGQPASISCKSSQSLLYSNGKTYLNWLLQKPGQSPQRLIYQVSKLDPGVPDRFSGSGSETDFTLKISRVEAEDVGVYYCMQGSSLPWTFGGGTKVEIKR

SEQ ID NO: 22 - protein, 113 aa, unidentified
DIVMTQTPLSLSVTIGQPASISCKSSQSLLYSNGKTYLNWLLQKPGQSPQRLIYQVSKLDPGVPDRFSGSGSETDFTLKISRVEAEDVGVYYCMQGSSLPWTFGGGTKVEIKR

SEQ ID NO: 23 - protein, 113 aa, unidentified
DIVMTQTPLSLSVTIGQPASISCKSSQSLLYSNGKTYLNWLLQKPGQSPKRLIYQVSKLDPGIPDRFSGSGSETDFTLKISRVEAEDVGIYYCMQGSSLPWTFGGGTKVEIKR

SEQ ID NO: 24 - protein, 113 aa, unidentified
DIVMTQTPLSLSVTIGQPASISCKSSQSLLYSNGKTYLNWLLQKPGQSPKRLIYQVSKLDPGVPDRFSGSGSETDFTLKISRVEAEDVGVYYCMQGSSLPWTFGGGTKVEIKR

SEQ ID NO: 25 - protein, 329 aa, unidentified
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 26 - protein, 106 aa, unidentified
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 8 aa, unidentified
SNSGLAPN